(19) United States
(12) Patent Application Publication
(10) Pub. No.: US 2026/0027289 A1
(43) Pub. Date: Jan. 29, 2026

(54) Drug Delivery Device With Bi-Directional Oscillo-Rotative Pump

(71) Applicant: Becton, Dickinson and Company, Franklin Lakes, NJ (US)

(72) Inventors: Steve Beguin, Nenzlingen (CH); David James Coleman, Shankill (IE); Elaine Huggard, Rathgar (IE); Katie Fisher, Dublin 18 (IE); Slobodan Stefanov, Deerfield Beach, FL (US)

(21) Appl. No.: 18/997,810

(22) PCT Filed: Oct. 10, 2023
(86) PCT No.: PCT/US2023/076416

§ 371 Date: Jan. 23, 2025

Related U.S. Application Data
(60) Provisional application No. 63/415,080, filed on Oct. 11, 2022.

(51) Int. Cl. A61M 5/142; A61M 5/145

(52) U.S. Cl. CPC A61M 5/14216; A61M 5/14248; A61M 5/1452

(57) ABSTRACT

A rotational metering pump for a drug delivery device having a reservoir and a downstream fluid path includes a sleeve defining a chamber, a manifold housing in fluid communication with the chamber, and a first valving system partially defined by the manifold housing. The first valving system is selectively in fluid communication with the chamber. The rotational metering pump further includes a second valving system partially defined by the manifold. The second valving system is selectively in fluid communication with the chamber and the first valving system.

### CROSS-REFERENCE TO RELATED APPLICATION

The present application claims priority to U.S. Provisional Application Ser. No. 63/415,080 entitled “Drug Delivery Device with Bi-Directional Oscillo-Rotative Pump” filed Oct. 11, 2022, the entire disclosure of which is hereby incorporated by reference in its entirety.

### BACKGROUND OF THE INVENTION

### Field of the Invention

The present disclosure relates generally to metering pumps for drug delivery devices.

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

Current automatic injectors utilize a pump architecture that provides unidirectional flow. Many applications require multi-directional flow, which requires the use of multiple pumps. Thus, there is a need for automatic injectors that provide multidirectional flow with a single pump. Accordingly, those skilled in the art continue with research and development efforts in the field of metering pumps for drug delivery devices.

### SUMMARY OF THE INVENTION

Disclosed is a rotational metering pump for a drug delivery device having a reservoir and a downstream fluid path.

In one example, the rotational metering pump includes a sleeve defining a chamber, a manifold housing in fluid communication with the chamber, and a first valving system partially defined by the manifold housing. The first valving system is selectively in fluid communication with the chamber. The rotational metering pump further includes a second valving system partially defined by the manifold housing. The second valving system is selectively in fluid communication with the chamber and the first valving system.

The manifold housing may house a first manifold in fluid communication with the first valving system and a second manifold in fluid communication with the second valving system.

The first valving system may include a first port and a second port, and the second valving system may include a third port and a fourth port. The first port of the first valving system and the third port of the second valving system may be in fluid communication with the reservoir. The second port of the first valving system and the fourth port of the first second system may be in fluid communication with the downstream fluid path. One of the first valving system and the second valving system may be in fluid communication with a fill port.

The rotational metering pump may include a piston at least partially received within the sleeve, the piston and the sleeve defining the chamber, the piston having a first position where the chamber has a first volume and a second position where the chamber has a second volume, the first volume larger than the second volume.

The sleeve may be rotatable about a central axis between at least a first rotational position and a second rotational position. The channel may be in fluid communication with the first port of the first valving system when the sleeve is in the first rotational position. The channel may be in fluid communication with the second port of the first valving system when the sleeve is in the second rotational position.

The sleeve may be rotatable about a central axis between at least a third rotational position and a fourth rotational position. The channel may be in fluid communication with the third port of the second valving system when the sleeve is in the third rotational position and the channel may be in fluid communication with the fourth port of the second valving system when the sleeve is in the fourth rotational position. The sleeve may further be rotatable to an isolated position such that the first valving system and the second valving system are isolated from the chamber.

The first valving system may include at least one elastomeric seal. Further, the second valving system may include at least one elastomeric seal.

The piston may be configured to rotate and axially move relative to the housing and the sleeve, and the piston may be configured to rotate together with the sleeve relative to the housing.

Rotation of the piston in a first rotational direction may be configured to aspirate a fluid within the chamber and move the sleeve from the first rotational position to the second rotational position, and rotation of the piston in a second rotational direction may be configured to pump a fluid within the chamber and move the sleeve from the second rotational position to the first rotational position, the second rotational direction being opposite from the first rotational direction.

In another configuration, the rotation of the piston in a third rotational direction is configured to aspirate a fluid within the chamber and move the sleeve from the third rotational position to the further rotations position. The rotation of the piston in a further rotational direction is configured to pump a fluid within the chamber and move the sleeve from the fourth rotational position to the third rotational position, with the further rotational direction being opposite from the third rotational direction.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1A is an exploded view of a rotating metering pump for a drug delivery device;

FIG. 1B is an exploded view of a rotating metering pump for a drug delivery device;

FIG. 2 is a perspective view of a rotational metering pump;

FIG. 3 is a perspective cross-sectional view of a portion of the rotational metering pump of FIG. 2;

FIG. 4A is a schematic of a portion of a drug delivery device;

FIG. 4B is a schematic of a portion of a drug delivery device; and

FIG. 5 is schematic of a series of fluid movement within the rotational metering pump of FIG. 2.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

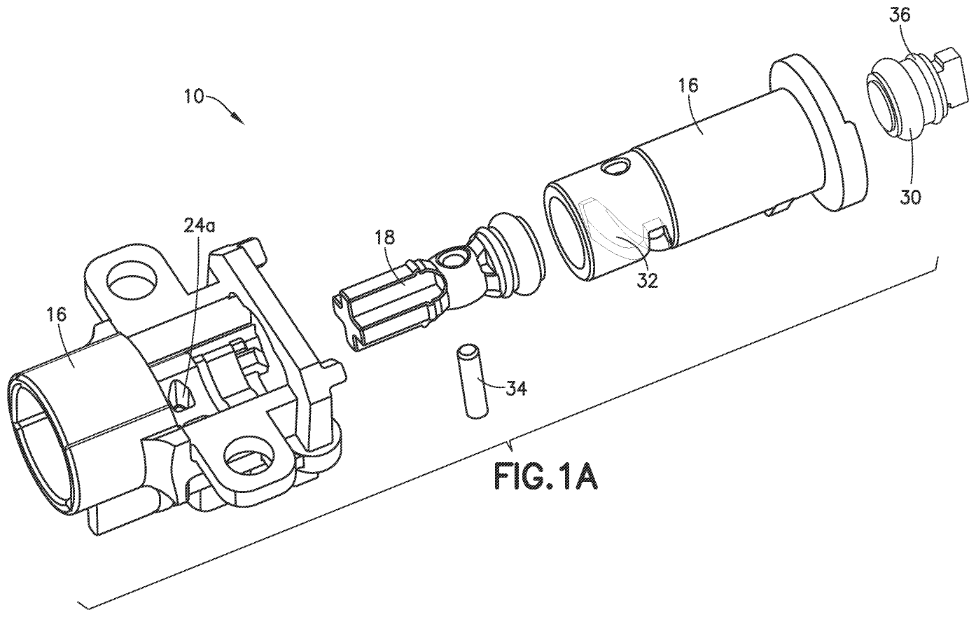

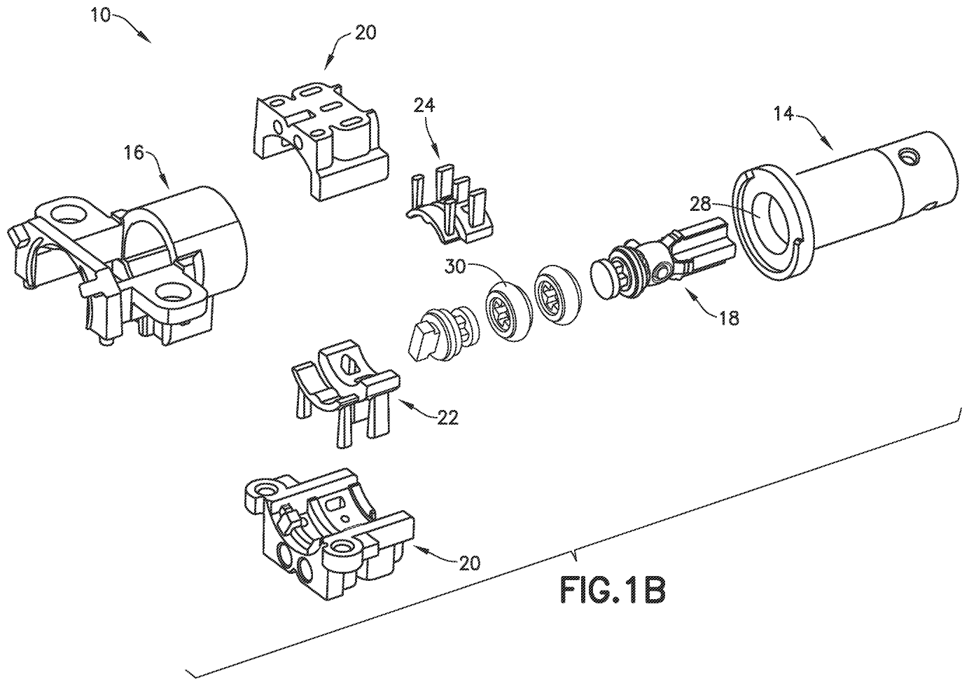

Referring generally to FIG. 1A and FIG. 1B, a metering pump 10 for a medical injector or drug delivery device 12 is shown. The metering pump 10 is a rotational metering pump, which is described in International Publication No. WO 2015/157174, which is hereby incorporated by reference in its entirety. The rotational metering pump 10 is configured to be connected to a DC motor and gearbox assembly (not shown) to rotate a sleeve 14 in a housing 16. A helical groove 32 is provided on the sleeve 14. A coupling pin 34 connected to a piston 18 translates along the helical groove 32 to guide the retraction and insertion of the piston 18 within the sleeve 14, respectively, as the sleeve 14 rotates in one direction and then rotates in the opposite direction. The sleeve 14 has an end plug 36. Two seals 30 on the respective ends of the piston 18 and the end plug 36 that are interior to the sleeve 14 define a cavity or chamber 28 when the piston 18 is retracted, following an aspirate stroke and, therefore, ready to dispense. The volume of the chamber 28 changes depending on the degree of retraction of the piston 18. The volume of the chamber 28 is negligible or essentially zero when the piston 18 is fully inserted and the seals 30 are substantially in contact with each other following a dispense stroke, and therefore ready to aspirate.

In one example, the disclosed rotational metering pump 10 may be characterized as an oscillo-rotative pump 10 with at an integrated valving architecture having at least two valving systems 22, 24. The disclosed architecture of the rotational metering pump 10 enables pumping of fluid through different channels independently by way of a single pump. One exemplary application for the disclosed pump 10 is the use of the rotational metering pump 10 to fill a reservoir 6 of a drug delivery device 12 and enable a vial to device transfer system via fill port 4, while using a single motor to control the valving systems and actuate the rotational metering pump 10.

A series of ports 22a, 22b, 24a, and 24b are provided relative to the housing 16 via manifold housing 20. Each port 22a, 22b, 24a, and 24b is configured such that fluid, such as medication, can flow from a fill port 4 to the rotational metering pump 10, and then to a reservoir 6. The fluid can then move back to the rotational metering pump 10 and out a different port through which the fluid or medication has been drawn into the chamber 28 (e.g., by retraction of the piston 18 during an aspirate stage of operation). The fluid can be dispensed from the chamber 28 to, for example, a fluid path to a downstream fluid path 8, such as a cannula, in the patient by re-insertion of the piston 18 into the chamber 28.

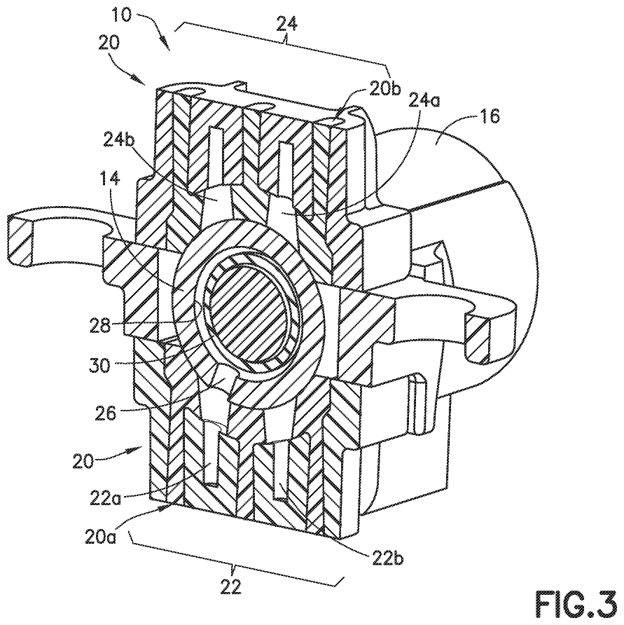

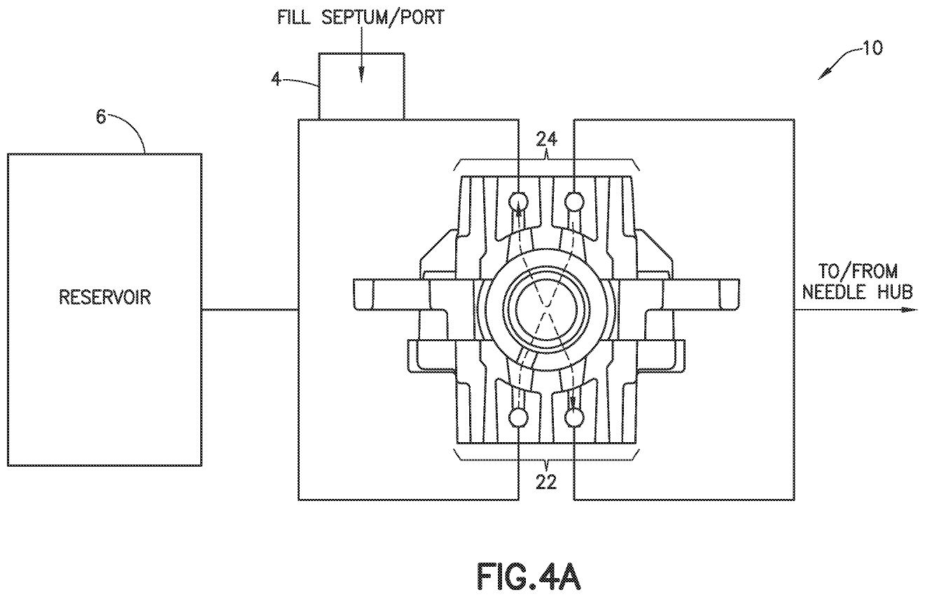

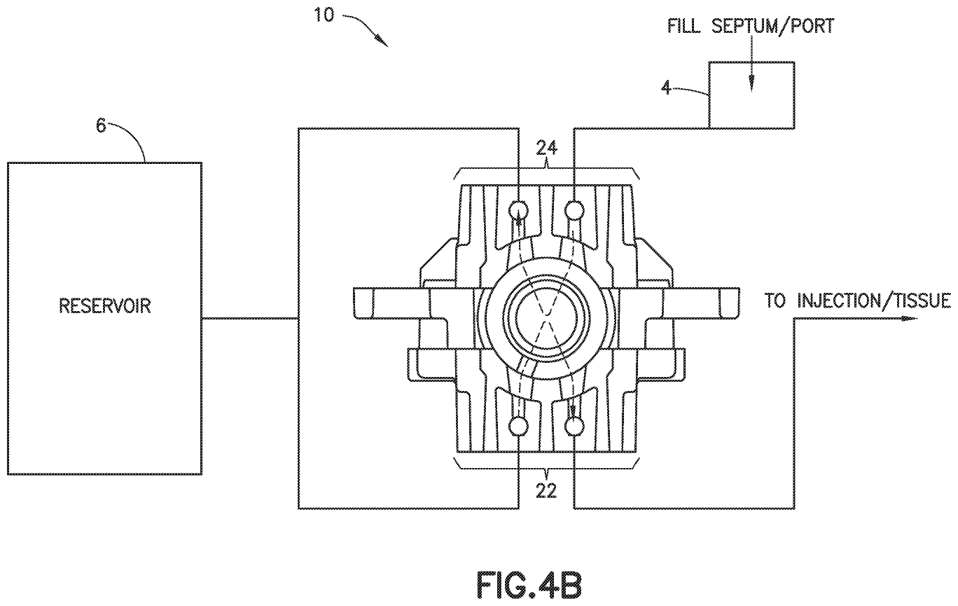

Referring generally to FIGS. 1-5, the disclosed rotational metering pump 10 is configured for use in a drug delivery device 12, such as a medical injector. The drug delivery device 12 includes a reservoir 6 and a downstream fluid path 8, such as a cannula, see FIG. 4A and FIG. 4B. The rotational metering pump 10 includes a housing 16, see FIG. 1B. The housing 16 defines a chamber 28 configured to receive a sleeve 14.

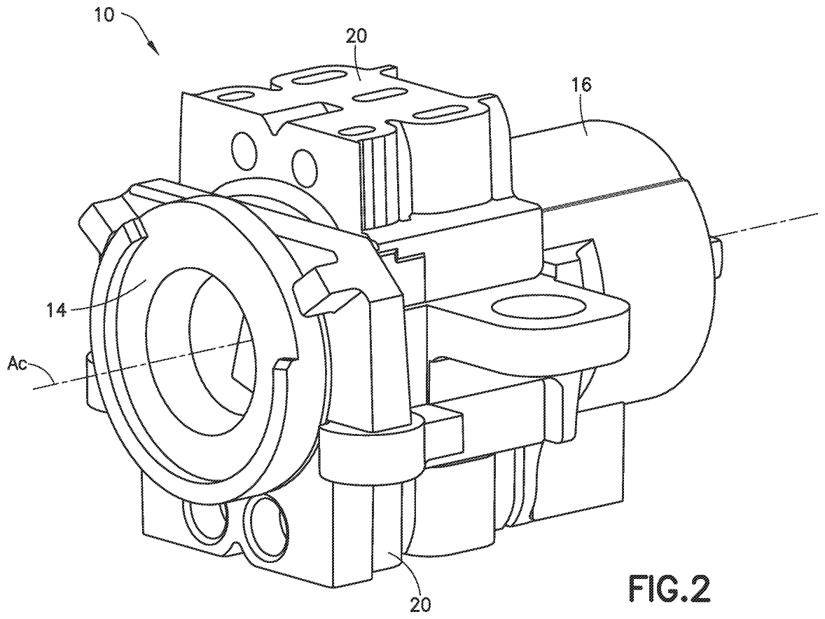

Referring to FIG. 1B, the rotational metering pump 10 includes a manifold housing 20 coupled to the housing 16. The manifold housing 20 is configured to be in fluid communication with the chamber 28 and two or more valving systems 22, 24 for selective passage of fluid, such as medication. In one example, the manifold housing 20 partially defines a first valving system 22, see FIG. 2, in fluid communication with a first manifold 20a. The first valving system 22 is selectively in fluid communication with the chamber 28 based upon alignment of features within the rotational metering pump 10 explained below. The first valving system 22 comprises a first port 22a and a second port 22b. It is understood that the first port 22a may also be referred to as a manifold seal inlet 22a and the second port 22b may be referred to as a manifold seal outlet 22b. In one example, the first valving system 22 comprises at least one elastomeric seal 30. The elastomeric seal 30 may be over-molded.

Still referring to FIG. 2, the manifold housing 20 partially defines a second valving system 24 in fluid communication with a second manifold 20b. The second valving system 24 is selectively in fluid communication with the chamber 28 and the first valving system 22 based upon alignment of features within the rotational metering pump 10 explained below. The second valving system 24 comprises a third port 24a and a fourth port 24b. It is understood that the third port 24a may be referred to as a manifold seal inlet 24a and the fourth port 24b may be referred to as a manifold seal outlet 24b. In one example, the second valving system 24 comprises at least one elastomeric seal 30. The elastomeric seal 30 may be over-molded the piston 18.

Referring to FIGS. 4A and 4B, in one or more examples, the first port 22a of the first valving system 22 and the third port 24a of the second valving system 24 are in fluid communication with the reservoir 6. Further, the second port 22b of the first valving system 22 and the fourth port 24b of the first second system 24 are in fluid communication with the downstream fluid path 8. Even further, in one or more examples, one or more of the first port 22a and the second port 22b of first valving system 22 and/or the third port 24a and the fourth port 24b of the second valving system 24 may be in fluid communication with a fill port 4 for filling the reservoir 6 with fluid, such as medication.

Referring back to FIG. 2, the rotational metering pump 10 further includes a sleeve 14 at least partially received within the housing 16. The sleeve 14 is rotatable about a central axis A_{C}. Rotation of the sleeve 14 may be facilitated by the piston 18. The sleeve 14 defines a channel 26 configured to align with the first port 22a, second port 22b, third port 24a, and fourth port 24b based upon rotation of the sleeve 14 about the central axis A_{C}.

The sleeve 14 is rotatable 360° about the central axis A_{C} between a plurality of positions. In one example, the sleeve 14 is rotatable about the central axis A_{C} between at least a first rotational position and a second rotational position. The channel 26 is in fluid communication with the first port 22a of the first valving system 22 when the sleeve 14 is in the first rotational position. Further, the channel 26 is in fluid communication with the second port 22b of the first valving system 22 when the sleeve 14 is in the second rotational position.

Additionally, the sleeve 14 is may be rotatable about the central axis A_{C} between at least a third rotational position and a fourth rotational position. When the sleeve 14 is in the third rotational position, the channel 26 is in fluid communication with the third port 24a of the second valving system 24. When the sleeve 14 is in the fourth rotational position, the channel 26 is in fluid communication with the fourth port 24b of the second valving system 24.

The sleeve 14 is further configured to rotate to an isolated position such that the first valving system 22 and the second valving system 24 are isolated from the chamber 28. The isolated position may be located between the first valving system 22 and the second valving system 24 or may be located between ports of one of the first valving system 22 and the second valving system 24. An isolated position may be anywhere such that the channel 26 is not aligned with a port and therefore is not in fluid communication with the first valving system 22 or the second valving system 24.

The manifold housing 20 may include a sealing surface that is in continuous contact with an outer surface of the sleeve 14 such that the manifold housing 20 maintains continuous closing of the chamber 28 during rotation of the sleeve 14 between manifold ports so as to keep a fluid tight seal.

Referring to FIG. 1B, in one or more examples, the rotational metering pump 10 further includes a piston 18 at least partially received within the sleeve 14. The piston 18 and the sleeve 14 define the chamber 28 within the housing 16. The piston 18 in moveable between at least a first position where the chamber 28 has a first volume and a second position where the chamber 28 has a second volume, wherein the first volume larger than the second volume. The piston 18 is configured to rotate and axially move relative to the housing 16 and the sleeve 14. In one example, the piston 18 is configured to rotate together with the sleeve 14 relative to the housing 16. In another example, the piston 18 is configured to rotate independently from the sleeve 14 relative to the housing 16.

In one non-limiting example, rotation of the piston 18 in a first rotational direction is configured to aspirate a fluid within the chamber 28 and move the sleeve 14 from the first rotational position to the second rotational position. Further rotation of the piston 18 in a second rotational direction is configured to pump a fluid within the chamber 28 and move the sleeve 14 from the second rotational position to the first rotational position, the second rotational direction being opposite from the first rotational direction.

Referring to FIGS. 1 and 2, in one or more examples, the piston 18 is configured to rotate and axially move relative to the housing 16 and the sleeve 14, with the piston 18 configured to rotate together with the sleeve 14 relative to the housing 16 to allow pumping of fluid in multiple channels defined by the manifold housing 20. The piston 18 is connected to the sleeve 14 via a coupling pin 34 received within a helical groove 32 defined by the sleeve 14. The first valving system 22 and the second valving system 24 are configured to selectively be in fluid communication with both the reservoir 6 of the drug delivery device 12 and the downstream fluid path 8 of the drug delivery device 12 via rotation of the sleeve 14.

The first valving system 22 and/or the second valving system 24 may further be in fluid communication with a fill port 4 for filling the reservoir 6 with fluid, such as medication. Rotation of the piston 18 in a first rotational direction is configured to aspirate a fluid within the chamber 28 and move the sleeve 14 from the first rotational position to the second rotational position, and rotation of the piston 18 in a second rotational direction is configured to pump a fluid within the chamber 28 and move the sleeve 14 from the second rotational position to the first rotational position, the second rotational direction being opposite from the first rotational direction. Further, rotation of the piston 18 in a third rotational direction is configured to aspirate a fluid within the chamber 28 and move the sleeve 14 from the third rotational position to the fourth rotational position, and wherein rotation of the piston 18 in a fourth rotational direction is configured to pump a fluid within the chamber 28 and move the sleeve 14 from the fourth rotational position to the third rotational position, the fourth rotational direction being opposite from the third rotational direction.

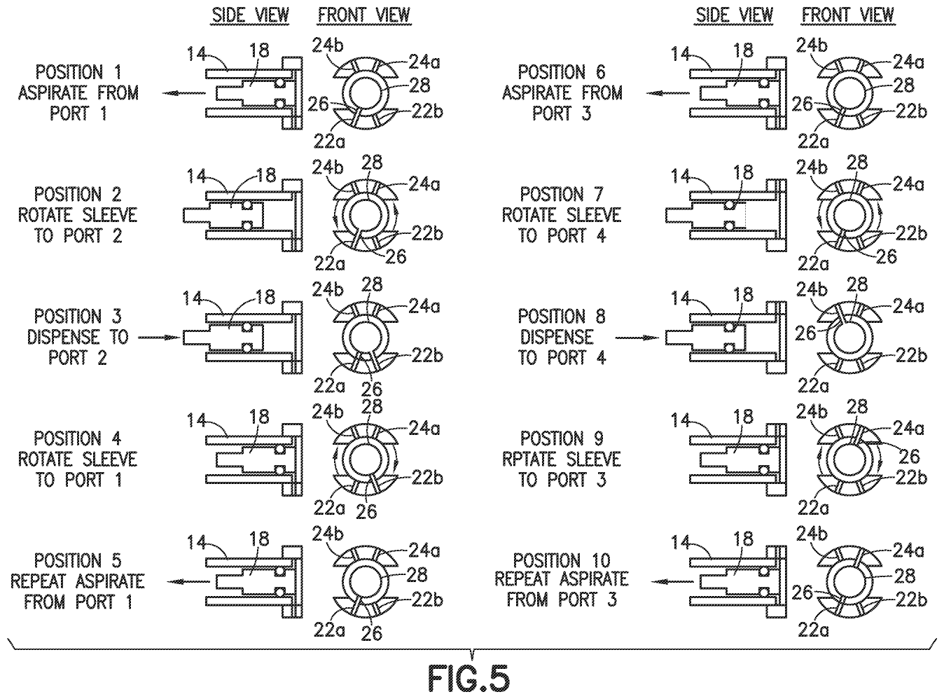

FIG. 5 illustrates, in a series of exemplary schematics, how rotation and movement of the piston 18 and sleeve 14 facilitate movement of fluid within the rotational metering pump 10. The rotational metering pump 10 may be configured such that fluid may be pumped throughout the first valving system 22 from manifold seal inlet or first port 22a to a manifold seal outlet or second port 22b and the second valving system 24 from a manifold seal inlet or third port 24a to a manifold seal outlet or fourth port 24b via the chamber 28. The exemplary schematics illustrated in FIG. 5 are merely one example of how fluid may flow throughout a fluid circuit via the rotational metering pump 10. The flow of fluid thought out the rotational metering pump 10 is not limited to the schematic sequence illustrated in FIG. 5.

As illustrated in the example of FIG. 5, movement of the piston 18 between position1 and position 2 facilitates aspiration of fluid from the first valving system 22 via the first port 22a. At position 1 and position 2, the sleeve 14 is positioned such that the channel 26 is aligned with the first port 22a, thus placing the chamber 28 in fluid communication with the first port 22a. In one example, the first port 22a is concurrently in fluid communication with the reservoir 6 such that fluid may travel between the reservoir 6 and the chamber 28 via the first port 22a and the channel 26 based upon movement of the piston 18. Retraction and insertion of the piston 18 within the sleeve 14 may occur concurrently with rotation of the piston 18 and/or sleeve 14 or may occur independently from rotation of the piston and/or sleeve.

Still referring to FIG. 5, position 3 and position 4 illustrate movement of fluid from the chamber 28 through the second port 22b via the channel 26. The sleeve 14 rotates or oscillates about the central axis A_{C} such that the channel 26 aligns with the second port 22b of the first valving system 22, thus placing the second port 22b in fluid communication with the chamber 28. In one example, the second port 22b is concurrently in fluid communication with the downstream fluid path 8. In another example, the second port 22b is concurrently in fluid communication with an injection site, or needle hub, sec FIGS. 4A and 4B. The aspiration from the first port 22a may then be repeated, as illustrated in position 5.

Upon completion of positions 1-5, in one or more examples, the sleeve 14 may rotate to an orientation such that the channel 26 is in proximity to the third port 24a and the fourth port 24b to facilitate alignment with the third port 24a and the fourth port 24b. Referring to position 6 of FIG. 4, the piston 18 may retract so as to aspirate fluid through the third port 24a. The third port 24a may be in fluid communication with a fill port 4 so as to draw fluid from the fill port 4 to the chamber 28 for transfer to the reservoir 6 via the fourth port 24b, see position 7. Position 8 illustrates dispensing of the fluid from the chamber 28 to the reservoir 6 via the fourth port 24b by inserting the piston 18 into the sleeve 14. The sleeve 14 may then rotate or oscillate to an orientation such that the channel 26 is aligned with the first port 24a again and that aspiration/dispensing positions 6-8 may be repeated.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A rotational metering pump for a drug delivery device comprising a reservoir and a downstream fluid path, the rotational metering pump comprising:
a sleeve defining a chamber;
a manifold housing fluid communication with the chamber;
a first valving system partially defined by the manifold housing, the first valving system selectively in fluid communication with the chamber; and
a second valving system partially defined by the manifold housing, the second valving system selectively in fluid communication with the chamber and the first valving system, wherein the first valving system comprises a first port and a second port: and
the second valving system comprises a third port and a fourth port.

2. The rotational metering pump of claim 1, wherein the manifold housing houses a first manifold in fluid communication with the first valving system and a second manifold in fluid communication with the second valving system.

3. (canceled)

4. The rotational metering pump of claim 1, 3, wherein the manifold housing comprises a sealing surface in continuous contact with an outer surface of the sleeve such that the manifold housing maintains continuous closing of the chamber during rotation of the sleeve between manifold ports.

5. The rotational metering pump of claim 1, wherein the first port of the first valving system and the third port of the second valving system are in fluid communication with the reservoir.

6. The rotational metering pump of claim 1, wherein the second port of the first valving system and the fourth port of the first second system are in fluid communication with the downstream fluid path.

7. The rotational metering pump claim 1, further comprising:
a piston at least partially received within the sleeve, the sleeve defining a channel, the piston and the sleeve defining the chamber, the piston having a first position where the chamber has a first volume and a second position where the chamber has a second volume, the first volume larger than the second volume.

8. The rotational metering pump of claim 6, wherein:
the sleeve is rotatable about a central axis (A_{C}) between at least a first rotational position and a second rotational position;
the channel is in fluid communication with the first port of the first valving system when the sleeve is in the first rotational position; and
the channel is in fluid communication with the second port of the first valving system when the sleeve is in the second rotational position.

9. The rotational metering pump of claim 6, wherein:
the sleeve is rotatable about a central axis (A_{C}) between at least a third rotational position and a fourth rotational position;
the channel is in fluid communication with the third port of the second valving system when the sleeve is in the third rotational position; and
the channel is in fluid communication with the fourth port of the second valving system when the sleeve is in the fourth rotational position.

10. The rotational metering pump of claim 6, wherein the sleeve is rotatable to an isolated position such that the first valving system and the second valving system are isolated from the chamber.

11. The rotational metering pump of claim 6, wherein the first valving system comprises at least one elastomeric seal.

12. The rotational metering pump of claim 6, wherein the second valving system comprises at least one elastomeric seal.

13. The rotational metering pump of claim 6, wherein at least one of the first valving system and the second valving system is in fluid communication with a fill port.

14. The rotational metering pump of claim 6, wherein the piston is configured to rotate and axially move relative to the housing and the sleeve, and wherein the piston is configured to rotate together with the sleeve relative to the housing.

15. The rotational metering pump of claim 13, wherein rotation of the piston in a first rotational direction is configured to aspirate a fluid within the chamber and move the sleeve from the first rotational position to the second rotational position, and wherein rotation of the piston in a second rotational direction is configured to pump a fluid within the chamber and move the sleeve from the second rotational position to the first rotational position, the second rotational direction being opposite from the first rotational direction.

16. The rotational metering pump of claim 13, wherein rotation of the piston in a third rotational direction is configured to aspirate a fluid within the chamber and move the sleeve from the third rotational position to the fourth rotational position, and wherein rotation of the piston in a fourth rotational direction is configured to pump a fluid within the chamber and move the sleeve from the fourth rotational position to the third rotational position, the fourth rotational direction being opposite from the third rotational direction.
